(19) United States
(12) Patent Application Publication
(10) Pub. No.: US 2023/0265196 A1
(43) Pub. Date: Aug. 24, 2023

(54) Combination Therapy of a PD-1 Antagonist and an Antagonist for VEGFR-2 for Treating Patients with Cancer

(71) Applicants: PharmAbcine Inc., Daejeon (KR); Merck Sharp & Dohme B.V., Haarlem (NL); MSD International GmbH, Luzern (CH)

(72) Inventors: Seon Young Lee, Seoul (KR); Weon Sup Lee, Daejeon (KR); Jin-San Yoo, Daejeon (KR); Sang Ryeol Shim, Daejeon (KR)

(21) Appl. No.: 18/043,602

(22) PCT Filed: Sep. 2, 2021
(86) PCT No.: PCT/IB2021/058043

§ 371 Date: Mar. 1, 2023

Related U.S. Application Data
(60) Provisional application No. 63/122,321, filed on Dec. 7, 2020.
(60) Provisional application No. 63/073,512, filed on Sep. 2, 2020.

(51) Int. Cl. C07K 16/28; A61P 35/04; A61P 35/00

(52) U.S. Cl. CPC C07K 16/2863; C07K 16/2818; A61P 35/04; A61P 35/00; C07K 2317/24; C07K 2317/21

(57) ABSTRACT

The present disclosure describes combination therapies comprising an antagonist of Programmed cell Death 1 receptor (PD-1) and a vascular endothelial growth factor receptor-2 (VEGFR-2) antagonist, and the use of the combination therapies for the treatment of a cancer. In an embodiment, the cancer is a glioblastoma, a breast cancer, a triple negative breast cancer, a metastatic breast cancer, or a metastatic triple negative breast cancer.

### CROSS REFERENCE TO RELATED APPLICATIONS

This application claims benefit of U.S. Provisional Application No. 63/122,321, filed Dec. 7, 2020 and U.S. Provisional Application No. 63/073,512, filed Sep. 2, 2020, which is hereby incorporated in its entirety for all purposes.

### SEQUENCE LISTING

The instant application contains a Sequence Listing that has been submitted electronically in ASCII format and is hereby incorporated by reference in its entirety. Said ASCII copy, created on Aug. 13, 2021, is named 222870_0001-WO-000003 SL.txt and is 28,180 bytes in size.

### FIELD OF THE INVENTION

The present invention relates to combination therapies useful for the treatment of cancer. In particular, the invention relates to a combination therapy that comprises an antagonist of a Programmed cell death protein 1 (PD-1) and an antagonist of vascular endothelial growth factor receptor 2 (VEGFR-2).

### BACKGROUND

PD-1 is recognized as an important molecule in immune regulation and the maintenance of peripheral tolerance. PD-1 is moderately expressed on naive T, B and NKT cells and up-regulated by TB cell receptor signaling on lymphocytes, monocytes and myeloid cells (1).

Two known ligands for PD-1, PD-L1 (B7-H1) and PD-L2 (B7-DC), are expressed in human cancers arising in various tissues. In large sample sets of e.g. ovarian, renal, colorectal, pancreatic and liver cancers, and melanoma, it was shown that PD-L1 expression correlated with poor prognosis and reduced overall survival irrespective of subsequent treatment (2-13). Similarly, PD-1 expression on tumor infiltrating lymphocytes was found to mark dysfunctional T cells in breast cancer and melanoma (14-15) and to correlate with poor prognosis in renal cancer (16). Thus, it has been proposed that PD-L1 expressing tumor cells interact with PD-1 expressing T cells to attenuate T cell activation and evasion of immune surveillance, thereby contributing to an impaired immune response against the tumor.

Several monoclonal antibodies that inhibit the interaction between PD-1 and one or both of its ligands PD-L1 and PD-L2 have been approved for treating cancer. Pembrolizumab is a potent humanized immunoglobulin G4 (IgG4) mAb with high specificity of binding to the programmed cell death 1 (PD 1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD-1. KEYTRUDA® (pembrolizumab; Merck Sharp & Dohme, Corp., Rahway, N.J.) is indicated and stands approved for the treatment of patients across a number of indications.

Tumor angiogenesis is essential for cancer cell growth and survival with supply of the required oxygen and nutrients and may be important in the metastatic process. Vascular endothelial growth factor (VEGF) is a central regulator of vasculogenesis in embryonic development and angiogenesis in adults. In many human tumors including lung, breast, gastrointestinal tract, kidney and ovarian carcinomas, the expression of VEGF is potentiated by a variety of cytokines, growth factors (e.g., fibroblast growth factor (FGF) or platelet-derived growth factor (PDGF)) or by activated oncogenes.

VEGF induces angiogenesis by binding to a cell surface receptor named VEGF receptor (VEGFR). VEGFR is a representative receptor tyrosine kinase (RTK), which enhances proliferation, growth, and differentiation of endothelial cells. The VEGFR exists as 3 isoforms, VEGFR-1, VEGFR-2 and VEGFR-3, also known as Flt-1, KDR (Flt-1 in mouse) and Flt-4, respectively. Among them, VEGFR-2/KDR is one of the key receptors in endothelial cell proliferation and angiogenesis by VEGF signaling, and it is known that VEGFR-2/KDR can regulate cell growth through an autocrine pathway.

Once VEGF binds to VEGFR-2/KDR undergoes auto-phosphorylation and engages key downstream pathways, including PI3K-AKT and the RAS-RAF-MEK-MAPK signalling network, which are essential for stimulating diffusion, migration and survival of endothelial cell.

Olinvacimab (also known as TTAC-0001) binds to VEGFR-2 with high affinity, therefore effectively blocking VEGF binding to VEGFR-2/KDR The blockade of VEGF binding to VEGFR-2/KDR results in the inhibition of VEGFR-2 phosphorylation and downstream signalling. Consequently, olinvacimab presents anti-tumor as well as anti-angiogenesis effects.

Breast cancer is the most common cancer among women in the United States, the second most common cause of cancer death, and the main cause of death in women ages 45 to 55 years. Triple-negative breast cancer (TNBC) accounts for approximately 15% to 20% of breast cancers. TNBC is a subtype of breast cancer defined classically by its lack of estrogen receptor, progesterone receptor, and human epidermal growth factor receptor 2 (HER2) overexpression, thereby making it difficult to target. Compared to other breast cancer subtypes, TNBC tumors are frequently larger, less differentiated and approximately 2.5-fold more likely to metastasize within 5 years of diagnosis. Median time to death for a patient with TNBC is therefore shorter (4.2 versus 6 years); and overall survival (OS) also is poorer for patients with TNBC compared to other breast cancers. Metastatic TNBC (mTNBC) represents a continuing challenge because when compared to other breast cancer subtypes, it is associated with a higher frequency of progression, shorter progression free survival (PFS) and poorer OS. The lung, bone, liver and brain are the most common metastatic target sites for breast cancer. In fact, approximately 60% of metastatic breast cancer patients suffer lung or bone metastasis in their life. The only currently available strategy for recurrent or metastatic triple-negative breast cancer (mTNBC) is re-challenging with systemic chemotherapy.

There remains an on-going need for cancer therapies, including for breast cancer, triple negative breast cancer, metastatic breast cancer, and metastatic triple negative breast cancer.

### SUMMARY OF THE INVENTION

In one embodiment, the invention provides a method for treating cancer in an individual comprising administering to the individual a combination therapy that comprises a PD-1 antagonist and a VEGFR-2 antagonist. In one embodiment, the cancer is triple-negative breast cancer (TNBC). In one embodiment, the TNBC is metastatic triple-negative breast cancer (mTNBC). In one embodiment, the individual has mTNBC with at least one metastatic lesion in the lung or brain. In one embodiment, the PD-1 antagonist and the VEGFR-2 antagonist are co-formulated. In another embodiment, the PD-1 antagonist and the VEGFR-2 antagonist are co-administered.

In one embodiment, the PD-1 antagonist is an anti-PD-1 antibody that blocks the binding of PD-1 to PD-L1 and PD-L2. In another embodiment, the VEGFR-2 antagonist is an anti-VEGFR-2 antibody that blocks the binding of VEGFR-2 to vascular endothelial growth factor (VEGF).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a graph of tumor size change from baseline as a function of treatment cycle for four patients with metastatic triple negative breast cancer in a clinical trial. Tumor size change was assessed using RECIST 1.1 criteria. The patients were treated with olinvacimab 12 mg/kg weekly (q7d) infusion in combination with pembrolizumab 200 mg day 1 in 3 week (q21d) cycles. PR=partial remission. PD=progressive disease. Solid star indicates partial response.

FIG. 2 depicts a graph of tumor size change from baseline as a function of treatment cycle for six patients with metastatic triple negative breast cancer in a clinical trial. Tumor size change was assessed using RECIST 1.1 criteria. The patients were treated with olinvacimab 16 mg/kg weekly (q7d) infusion in combination with pembrolizumab 200 mg day 1 in 3 week (q21d) cycles. CR=complete response. Solid star indicates partial response.

FIG. 3 depicts a graph (in the form of a “swimmer's plot”) of the interim results for patients with metastatic triple negative breast cancer in a clinical trial, as a function of treatment cycles. PR=partial remission. PD=progressive disease. SD=stable disease. Solid star indicates partial response. P=pembrolizumab. 0=olinvacimab. Patients were assessed after each second (even) treatment cycle. The numbers on the y-axis are the patient identifier.

FIG. 4 depicts CT scans of the lungs of patient 2202 before (left panel) and after (right panel) treatment with olinvacimab 16 mg/kg in combination with pembrolizumab. The left panel depicts a metastatic lung lesion (circled). The right panel depicts the lung after treatment with olinvacimab 16 mg/kg weekly (q7d) infusion in combination with pembrolizumab 200 mg day 1 in 3 week (q21d) cycles. The metastatic lung lesion is not observed in the right panel. The patient's lung lesion was measured and assessed according to RECIST 1.1 criteria.

### DETAILED DESCRIPTION

Abbreviations. Throughout the detailed description and examples of the invention the following abbreviations will be used:
BOR Best overall response
BID One dose twice daily
CBR Clinical Benefit Rate
CDR Complementarity determining region
CHO Chinese hamster ovary
CR Complete Response
DCR Disease Control Rate
DFS Disease free survival
DLT Dose limiting toxicity
DOR Duration of Response
DSDR Durable Stable Disease Rate
FFPE Formalin-fixed, paraffin-embedded
FR Framework region
IgG Immunoglobulin G
IHC Immunohistochemistry or immunohistochemical
irRC Immune related response criteria
IV Intravenous
MTD Maximum tolerated dose
NCBI National Center for Biotechnology Information
NCI National Cancer Institute
ORR Objective response rate
OS Overall survival
PD Progressive disease
PD-1 Programmed Cell Death Protein 1
PD-L1 Programmed Cell Death 1 Ligand 1
PD-L2 Programmed Cell Death 1 Ligand 2
PFS Progression free survival
PR Partial response
Q2W One dose every two weeks
Q3W One dose every three weeks
QD One dose per day
RECIST Response Evaluation Criteria in Solid Tumors
RTK Receptor tyrosine kinase
SD Stable disease
VEGF Vascular endothelial growth factor
VEGFR Vascular endothelial growth factor receptor
VH Immunoglobulin heavy chain variable region
VK Immunoglobulin kappa light chain variable region

### I. DEFINITIONS

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Administration” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “subject” includes any organism, preferably an animal, more preferably a mammal (e.g., rat, mouse, dog, cat, rabbit) and most preferably, primates and humans.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multi specific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883. As used herein, “VH” refers to a variable domain of the heavy chain. “VL” refers to a variable domain of the light chain.

As used herein, unless otherwise indicated, “antibody fragment” or “antigen binding fragment of an antibody” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)2, and Fv fragments; diabodies; linear antibodies; single-chain antibody molecules, e.g., sc-Fv; nanobodies and multi specific antibodies formed from antibody fragments. Such antibody fragments can have similar biological activity but perhaps less immunogenicity to the subject to whom it is administered.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Co-administration” as used herein for agents, such as the PD-1 antagonist or VEGFR-2 antagonist, means that the agents are administered so as to have overlapping therapeutic activities, and not necessarily that the agents are administered simultaneously to the subject. The agents may or may not be in physical combination prior to administration (e.g., in the same intravenous bag). In an embodiment, the agents are administered to a subject simultaneously or at about the same time. For example, the anti-PD-1 antibody and the anti-VEGFR-2 antibody may be contained in separate vials, when in liquid solution, and then may be mixed into the same intravenous infusion bag or injection device, and administered simultaneously to the patient.

“Co-formulated” or “co-formulation” or “coformulation” or “coformulated” as used herein refers to at least two different antibodies or antigen binding fragments thereof that are formulated together and stored as a combined product in a single vial or vessel (for example an injection device) rather than being formulated and stored individually and then mixed before administration or separately administered. In one embodiment, the co-formulation contains two different antibodies or antigen binding fragments thereof.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Anti-tumor response” when referring to a cancer patient treated with a therapeutic regimen, such as a combination therapy described herein, means at least one positive therapeutic effect, such as for example, reduced number of cancer cells, reduced tumor size, reduced rate of cancer cell infiltration into peripheral organs, reduced rate of tumor metastasis or tumor growth, or progression free survival. Positive therapeutic effects in cancer can be measured in a number of ways (see, W. A. Weber, J. Nucl. Med. 50: 1S-10S (2009); Eisenhauer et al., Eur. J Cancer 45: 228-247 (2009)). In some embodiments, an anti-tumor response to a combination therapy described herein is assessed using RECIST 1.1 criteria, bidimensional irRC or unidimensional irRC. In some embodiments, an anti-tumor response is any of SD, PR, CR, PFS, or DFS.

“Bidimensional irRC” refers to the set of criteria described in Wolchok J D, et al., “Guidelines for the evaluation of immune therapy activity in solid tumors: immune-related response criteria,” Clin. Cancer Res. 2009; 15(23): 7412-7420. These criteria utilize bidimensional tumor measurements of target lesions, which are obtained by multiplying the longest diameter and the longest perpendicular diameter (cm2) of each lesion.

“Biotherapeutic agent” means a biological molecule, such as an antibody or fusion protein, that blocks ligand/receptor signaling in any biological pathway that supports tumor maintenance and/or growth or suppresses the anti-tumor immune response. Classes of biotherapeutic agents include, but are not limited to, antibodies to EGFR, Her2/neu, other growth factor receptors, CD20, CD40, CD-40L, CTLA-4, OX-40, 4-1BB, and ICOS.

“CBR” or “Clinical Benefit Rate” means CR+PR+durable SD.

“CDR” or “CDRs” as used herein means complementarity determining region(s) in a immunoglobulin variable region, defined using the Kabat numbering system, unless otherwise indicated.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytoxic/antitumor antibiotics, topoisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, and anti-sense oligonucleotides that inhibit expression of genes implicated in abnormal cell proliferation or tumor growth. Chemotherapeutic agents useful in the treatment methods of the present invention include cytostatic and/or cytotoxic agents.

“Chothia” as used herein means an antibody numbering system described in A1-Lazikani et al., JMB 273: 927-948 (1997).

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met, Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a PD-1 antagonist that consists essentially of a recited amino acid sequence may also include one or more amino acids, including substitutions of one or more amino acid residues, which do not materially affect the properties of the binding compound.

“DCR” or “Disease Control Rate” means CR+PR+SD.“DSDR” or “Durable Stable Disease Rate” means SD for ≥23 weeks.

“Framework region” or “FR” as used herein means the immunoglobulin variable regions excluding the CDR regions.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.). “Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Non-responder patient”, when referring to a specific anti-tumor response to treatment with a combination therapy described herein, means the patient did not exhibit an anti-tumor response.

“ORR” or “objective response rate” refers in some embodiments to CR+PR; ORR (week 24) refers to CR and PR measured using irRECIST in each patient in a cohort after 24 weeks of anti-cancer treatment.

“Patient” or “subject” refers to any single subject for which therapy is desired or that is participating in a clinical trial, epidemiological study or used as a control, including humans, primates, and mammalian veterinary patients such as cattle, horses, dogs, and cats.

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc, and CD273 for PD-L2. In any of the treatment method, medicaments and uses of the present invention in which a human individual is being treated with a PD-1 antagonist, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

As used herein, an “olinvacimab variant” means a monoclonal antibody which comprises heavy chain and light chain sequences that are substantially identical to those in olinvacimab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine residue of the heavy chain. In other words, olinvacimab and a olinvacimab variant comprise identical CDR sequences, but differ from each other due to having a conservative amino acid substitution at no more than three or six other positions in their full length light and heavy chain sequences, respectively. An olinvacimab variant is substantially the same as olinvacimab with respect to the following properties: binding affinity to VEGFR-2 and ability to neutralize the vascular endothelial growth factor receptor (VEGFR)2/VEGF axis.

As used herein, a “pembrolizumab variant” means a monoclonal antibody which comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

“RECIST 1.1 Response Criteria” as used herein means the definitions set forth in Eisenhauer et al., E. A. et al., Eur. J Cancer 45: 228-247 (2009) for target lesions or nontarget lesions, as appropriate based on the context in which response is being measured.

“Responder patient” when referring to a specific anti-tumor response to treatment with a combination therapy described herein, means a patient exhibiting an anti-tumor response.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Tissue Section” refers to a single part or piece of a tissue sample, e.g., a thin slice of tissue cut from a sample of a normal tissue or of a tumor.

“Treat” or “treating” cancer as used herein means to administer a combination therapy of a PD-1 antagonist and a VEGFR-2 antagonist to a subject having cancer, or diagnosed with cancer, to achieve at least one positive therapeutic effect, such as for example, a reduced number of cancer cells, a reduced tumor size, a reduced rate of cancer cell infiltration into peripheral organs, or a reduced rate of tumor metastasis or tumor growth. Positive therapeutic effects in cancer can be measured in a number of ways (see, W. A. Weber, J. Nucl. Med. 50: 1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, response to a combination therapy described herein is assessed using RECIST 1.1 criteria or irRC (bidimensional or unidimensional) and the treatment achieved by a combination of the invention is any of PR, CR, OR, PFS, DFS and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a combination of the invention is any of PR, CR, PFS, DFS, OR and OS that is assessed using RECIST 1.1 response criteria. The treatment regimen for a combination of the invention that is effective to treat a cancer patient may vary according to factors such as the disease state, age, and weight of the patient, and the ability of the therapy to elicit an anti-cancer response in the subject. While an embodiment of any of the aspects of treating a subject with the described combination therapy may not be effective in achieving a positive therapeutic effect in every subject, it should do so in a statistically significant number of subjects as determined by any statistical test known in the art such as the Student's t-test, the chi2-test, the U-test according to Mann and Whitney, the Kruskal-Wallis test (H-test), Jonckheere-Terpstra-test and the Wilcoxon-test.

The terms “treatment regimen”, “dosing protocol” and “dosing regimen” are used interchangeably to refer to the dose and timing of administration of each therapeutic agent in a combination of the invention.

“Tumor” as it applies to a subject diagnosed with, or suspected of having, cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone marrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“VEGFR-2 antagonist” means any biological molecule that specifically binds to VEGFR-2 and blocks binding of VEGF to VEGFR-2 on an endothelial cell and inhibits VEGFR-2 phosphorylation. Inhibition of VEGFR-2 phosphorylation consequently inhibits downstream signaling, thereby neutralizing the VEGFR-2/VEGF axis and thereby blocking angiogenesis and inhibiting tumor growth and metastasis. Human VEGFR-2 comprises the amino acid sequence of Uniprot accession no. P35968. The N-terminal portion of the extracellular domain has the amino acid sequence shown in Table 2.

TABLE 2
Amino Acid Sequence of VEGFR2 Extracellular Domain ECD1~3

```
VEGFR2 (Extracellular domain ECD1-3)
                  10 20 30 40 50 60 70
....|....|....|....|....|....|....|....|....|....|....|....|....|....|
MQSKVLLAVALWLCVETRAASVGLPSVSLDLPRLSIQKDILTIKANTTLQITCRGQRDLDWLWPNNQSGS
                  80 90 100 100 120 130 140
....|....|....|....|....|....|....|....|....|....|....|....|....|....|
EQRVEVTECSDGLFCKTLTIPKVIGNDTGAYKCFYRETDLASVIYVYVQDYRSPFIASVSDQHGVVYITE
                150 160 170 180 190 200 210
....|....|....|....|....|....|....|....|....|....|....|....|....|....|
NKNKTVVIPCLGSISNLNVSLCARYPEKRFVPDGNRISWDSKKGFTIPSYMISYAGMVFCEAKINDESYQ
                220 230 240 250 260 270 280
....|....|....|....|....|....|....|....|....|....|....|....|....|....|
SIMYIVVVVGYRIYDVVLSPSHGIELSVGEKLVLNCTARTELNVGIDFNWEYPSSKHQHKKLVNRDLKTQ
                290 300 310 320
....|....|....|....|....|....|....|....|....|..
SGSEMKKFLSTLTIDGVTRSDQGLYTCAASSGLMTKKNSTFVRVHEK
(SEQ ID NO: 23; amino acids 1-327 of Uniprot accession no. P35968)
```

“Unidimensional irRC refers to the set of criteria described in Nishino M, Giobbie-Hurder A, Gargano M, Suda M, Ramaiya NH, Hodi FS. “Developing a Common Language for Tumor Response to Immunotherapy: Immune-related Response Criteria using Unidimensional measurements,” Clin Cancer Res. 2013, 19(14): 3936-3943). These criteria utilize the longest diameter (cm) of each lesion.

“Variable regions” or “V region” as used herein means the segment of IgG chains which is variable in sequence between different antibodies. Typically, it extends to Kabat residue 109 in the light chain and 113 in the heavy chain.

### PD-1 Antagonists and VEGFR-2 Antagonists

PD-1 antagonists useful in the treatment method, medicaments and uses of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

Examples of mAbs that bind to human PD-1, and useful in the treatment methods, medicaments and uses of the present invention, are described in U.S. Pat. Nos. 7,488,802, 7,521,051, 8,008,449, 8,354,509, 8,168,757, and PCT International application publ. Nos. WO2004/004771, WO2004/072286, WO2004/056875, and U.S. Patent publ. No. 2011/0271358. Specific anti-human PD-1 mAbs useful as the PD-1 antagonist in the treatment method, medicaments and uses of the present invention include: pembrolizumab (also known as MK-3475), a humanized IgG4 mAb with the structure described in WHO Drug Information, Vol. 27, No. 2, pages 161-162 (2013) and which comprises the heavy and light chain amino acid sequences shown in Table 3; nivolumab (BMS-936558), a human IgG4 mAb with the structure described in WHO Drug Information, Vol. 27, No. 1, pages 68-69 (2013) and which comprises the heavy and light chain amino acid sequences shown in Table 3; the humanized antibodies h409A11, h409A16 and h409A17, which are described in PCT International application publ. No. WO2008/156712, and AMP-514, which is being developed by MedImmune. Additional anti-PD-1 antibodies contemplated for use herein include MEDI0680 (U.S. Pat. No. 8,609,089), BGB-A317 (U.S. Patent publ. No. 2015/0079109), INCSHR1210 (SHR-1210) (PCT International application publ. No. WO2015/085847), REGN-2810 (PCT International application publ. No. WO2015/112800), PDR001 (PCT International application publ. No. WO2015/112900), TSR-042 (ANB011) (PCT International application publ. No. WO2014/179664) and STI-1110 (PCT International application publ. No. WO2014/194302).

Examples of mAbs that bind to human PD-L1 and are useful in the treatment methods, medicaments and uses of the present invention are described in PCT International application publ. Nos. WO2013/019906 and WO2010/077634 A1 and in U.S. Pat. No. 8,383,796. Specific anti-human PD-L1 mAbs useful as the PD-1 antagonist in the treatment methods, medicaments and uses of the present invention include MPDL3280A, BMS-936559, MEDI4736, MSB0010718C and an antibody that comprises the heavy chain and light chain variable regions of SEQ ID NO: 24 and SEQ ID NO: 21, respectively, of PCT International application publ. No. WO2013/019906.

Other PD-1 antagonists useful in the treatment methods, medicaments and uses of the present invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in PCT International application publ. Nos. WO2010/027827 and WO2011/066342. Specific fusion proteins useful as the PD-1 antagonist in the treatment methods, medicaments and uses of the present invention include AMP-224 (also known as B7-DCIg), which is a PD-L2-FC fusion protein and binds to human PD-1.

In some preferred embodiments of the treatment methods, medicaments and uses of the present invention, the PD-1 antagonist is a monoclonal antibody, or antigen binding fragment thereof, which comprises: (a) light chain CDR1, CDR2 and CDR3 of SEQ ID NOs: 1, 2 and 3, respectively, and (b) heavy chain CDR1, CDR2 and CDR3 of SEQ ID NOs: 6, 7 and 8, respectively.

In other preferred embodiments of the treatment methods, medicaments and uses of the present invention, the PD-1 antagonist is a monoclonal antibody, or antigen binding fragment thereof, which specifically binds to human PD-1 and comprises (a) a heavy chain variable region comprising SEQ ID NO: 9 or a variant thereof, and (b) a light chain variable region comprising SEQ ID NO: 4 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

In another preferred embodiment of the treatment methods, medicaments and uses of the present invention, the PD-1 antagonist is a monoclonal antibody that specifically binds to human PD-1 and comprises (a) a heavy chain comprising SEQ ID NO: 10 and (b) a light chain comprising SEQ ID NO: 5.

In yet another preferred embodiment of the treatment methods, medicaments and uses of the present invention, the PD-1 antagonist is a monoclonal antibody that specifically binds to human PD-1 and comprises (a) a heavy chain comprising SEQ ID NO: 12 and (b) a light chain comprising SEQ ID NO: 11. In one embodiment, the PD-1 antagonist is an anti-PD-1 antibody that comprises a heavy chain and a light chain, wherein the heavy and light chains comprise the amino acid sequences in SEQ ID NO: 10 and SEQ ID NO: 5, respectively.

In all of the above treatment methods, medicaments and uses, the PD-1 antagonist inhibits the binding of PD-L1 to PD-1, and preferably also inhibits the binding of PD-L2 to PD-1. In some embodiments of the above treatment methods, medicaments and uses, the PD-1 antagonist is a monoclonal antibody, or an antigen binding fragment thereof, which specifically binds to PD-1 or to PD-L1 and blocks the binding of PD-L1 to PD-1.

Table 3 below provides a list of the amino acid sequences of exemplary anti-PD-1 mAbs for use in the treatment methods, medicaments and uses of the present invention.

TABLE 3
Exemplary PD-1 Antibody Sequences

| | | |
|---|---|---|
| Antibody | | SEQ ID |
| Feature | Amino Acid Sequence | NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERATLSCRASKGVSTSGYSYLHW | 4 |
| Region | YQQKPGQAPRLLIYLASYLESGVPARFSGSGSGTDFTLT | |
| | ISSLEPEDFAVYYCQHSRDLPLTFGGGTKVEIK | |
| Light Chain | EIVLTQSPATLSLSPGERATLSCRASKGVSTSGYSYLHW | 5 |
| | YQQKPGQAPRLLIYLASYLESGVPARFSGSGSGTDFTLT | |
| | ISSLEPEDFAVYYCQHSRDLPLTFGGGTKVEIKRTVAAP | |
| | SVFIFPPSDEQLKSGTASVVCLLNNFYPREAKVQWKVD | |
| | NALQSGNSQESVTEQDSKDSTYSLSSTLTLSKADYEKH | |
| | KVYACEVTHQGLSSPVTKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGASVKVSCKASGYTFTNYYMYW | 9 |
| Region | VRQAPGQGLEWMGGINPSNGGTNFNEKFKNRVTLTTD | |
| | SSTTTAYMELKSLQFDDTAVYYCARRDYRFDMGFDY | |
| | WGQGTTVTVSS | |
| Heavy | QVQLVQSGVEVKKPGASVKVSCKASGYTFTNYYMYW | 10 |
| Chain | VRQAPGQGLEWMGGINPSNGGTNFNEKFKNRVTLTTD | |
| | SSTTTAYMELKSLQFDDTAVYYCARRDYRFDMGFDY | |
| | WGQGTTVTVSSASTKGPSVFPLAPCSRSTSESTAALGCL | |
| | VKDYFPEPVTVSWNSGALTSGVHTFPAVLQSSGLYSLS | |
| | SVVTVPSSSLGTKTYTCNVDHKPSNTKVDKRVESKYGP | |
| | PCPPCPAPEFLGGPSVFLFPPKPKDTLMISRTPEVTCVVV | |
| | DVSQEDPEVQFNWYVDGVEVHNAKTKPREEQFNSTYR | |
| | VVSVLTVLHQDWLNGKEYKCKVSNKGLPSSIEKTISKA | |
| | KGQPREPQVYTLPPSQEEMTKNQVSLTCLVKGFYPSDI | |
| | AVEWESNGQPENNYKTTPPVLDSDGSFFLYSRLTVDKS | |
| | RWQEGNVFSCSVMHEALHNHYTQKSLSLSLGK | |

Nivolumab Light Chain

| | | |
|---|---|---|
| Light Chain | EIVLTQSPATLSLSPGERATLSCRASQSVSSYLAWYQQK | 11 |
| | PGQAPRLLIYDASNRATGIPARFSGSGSGTDFTLTISSLE | |
| | PEDFAVYYCQQSSNWPRTFGQGTKVEIKRTVAAPSVFI | |
| | FPPSDEQLKSGTASVVCLLNNFYPREAKVQWKVDNAL | |
| | QSGNSQESVTEQDSKDSTYSLSSTLTLSKADYEKHKVY | |
| | ACEVTHQGLSSPVTKSFNRGEC | |

Nivolumab Heavy Chain

| | | |
|---|---|---|
| Heavy | QVQLVESGGGVVQPGRSLRLDCKASGITFSNSGMHWV | 12 |
| Chain | RQAPGKGLEWVAVIWYDGSKRYYADSVKGRFTISRDN | |
| | SKNTLFLQMNSLRAEDTAVYYCATNDDYWGQGTLVT | |
| | VSSASTKGPSVFPLAPCSRSTSESTAALGCLVKDYFPEP | |
| | VTVSWNSGALTSGVHTFPAVLQSSGLYSLSSVVTVPSS | |
| | SLGTKTYTCNVDHKPSNTKVDKRVESKYGPPCPPCPAP | |
| | EFLGGPSVFLFPPKPKDTLMISRTPEVTCVVVDVSQEDP | |
| | EVQFNWYVDGVEVHNAKTKPREEQFNSTYRVVSVLTV | |
| | LHQDWLNGKEYKCKVSNKGLPSSIEKTISKAKGQPREP | |
| | QVYTLPPSQEEMTKNQVSLTCLVKGFYPSDIAVEWESN | |
| | GQPENNYKTTPPVLDSDGSFFLYSRLTVDKSRWQEGN | |
| | VFSCSVMHEALHNHYTQKSLSLSLGK | |

VEGFR-2 antagonists useful in the treatment methods, medicaments and uses of the present invention include a monoclonal antibody (mAb), or antigen binding fragment thereof, which specifically binds to VEGFR-2. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

In one embodiment, the anti-VEGFR-2 antibody is olinvacimab (TTAC-0001). Olinvacimab recognizes two epitopes in the extracellular domain of VEGFR2: amino acids 111-117 and 219-225 of SEQ ID NO: 23. Olinvacimab also recognizes VEGFR2 of mice and rats; the epitope of amino acids 219-225 is 100% identical to the corresponding sequence in VEGFR2 of mice and rats. In certain embodiments, the anti-VEGFR-2 antibody can comprise:

an immunoglobulin light chain comprising the amino acid sequence of SEQ ID NO: 13; and an immunoglobulin heavy chain comprising the amino acid sequence of SEQ ID NO: 14; or

a light chain variable region comprising the amino acid sequence SEQ ID NO: 15; and a heavy chain variable region comprising the amino acid sequence SEQ ID NO: 16; or

a light chain variable region comprising CDR1: (SEQ ID NO: 17); CDR2: (SEQ ID NO: 18); and CDR3: (SEQ ID NO: 19); and a heavy chain variable region comprising CDR1: (SEQ ID NO: 20); CDR2: (SEQ ID NO: 21); and CDR3: (SEQ ID NO: 22).

Table 4 below provides a list of the amino acid sequences of olinvacimab (an exemplary anti-VEGFR-2 mAbs) for use in the treatment methods, medicaments and uses of the present invention.

TABLE 4
Exemplary VEGFR-2 Antibody Sequences

| | | |
|---|---|---|
| Antibody | | SEQ ID |
| Feature | Amino Acid Sequence | NO. |

Olinvacimab Light Chain

| | | |
|---|---|---|
| CDR1 | RGDNLGDVNVH | 17 |
| CDR2 | YDADRPS | 18 |
| CDR3 | QVWDRTSEYV | 19 |
| Variable | NFMLTQPPSVSVSPGKTARITCRGDNLGDVNVHWY | 15 |
| Region | QQRPGQAPVLVMYYDADRPSGIPERFSGSNSGNTAT | |
| | LTISGVEAGDEADYYCQVWDRTSEYVFGTGTKVTV | |
| | LG | |
| Light Chain | SGVGSNFMLTQPPSVSVSPGKTARITCRGDNLGDVN | 13 |
| | VHWYQQRPGQAPVLVMYYDADRPSGIPERFSGSNS | |
| | GNTATLTISGVEAGDEADYYCQVWDRTSEYVFGTG | |
| | TKVTVLGGGASLVERSVAAPSVFIFPPSDEQLKSGTA | |
| | SVVCLLNNFYPREAKVQWKVDNALQSGNSQESVTE | |
| | QDSKDSTYSLSSTLTLSKADYEKHKVYACEVTHQGL | |
| | SSPVTKSFNRGEC | |

Olinvacimab Heavy Chain

| | | |
|---|---|---|
| CDR1 | SYWMH | 20 |
| CDR2 | EINPGNGHTNYNEKFKS | 21 |
| CDR3 | IWGPSLTSPFDY | 22 |
| Variable | QMQLVQSGAEVKKPGASVKLSCKASGYTFSSYWMH | 16 |
| Region | WVRQAPGQRLEWMGEINPGNGHTNYNEKFKSRVTI | |
| | TVDKSASTAYMELSSLRSEDTAVYYCAKIWGPSLTS | |
| | PFDYWGQGTLVTVSS | |
| Heavy Chain | AQPAMAQMQLVQSGAEVKKPGASVKLSCKASGYTF | 14 |
| | SSYWMHWVRQAPGQRLEWMGEINPGNGHTNYNEK | |
| | FKSRVTITVDKSASTAYMELSSLRSEDTAVYYCAKI | |
| | WGPSLTSPFDYWGQGTLVTVSSGLGGLASTKGPSVF | |
| | PLAPSSKSTSGGTAALGCLVKDYFPEPVTVSWNSGA | |
| | LTSGVHTFPAVLQSSGLYSLSSVVTVPSSSLGTQTYIC | |
| | NVNHKPSNTKVDKKVEPKSCDKTHTCPPCPAPELLG | |
| | GPSVFLFPPKPKDTLMISRTPEVTCVVVDVSHEDPEV | |
| | KFNWYVDGVEVHNAKTKPREEQYNSTYRVVSVLTV | |
| | LHQDWLNGKEYKCKVSNKALPAPIEKTISKAKGQPR | |
| | EPQVYTLPPSREEMTKNQVSLTCLVKGFYPSDIAVE | |
| | WESNGQPENNYKTTPPVLDSDGSFFLYSKLTVDKSR | |
| | WQQGNVFSCSVMHEALHNHYTQKSLSLSPGK | |

In some preferred embodiments of the treatment methods, medicaments and uses of the drug combination, the VEGFR-2 antagonist is a monoclonal antibody, or antigen binding fragment thereof, which comprises: (a) a light chain variable region comprising light chain CDR1, CDR2 and CDR3 as set forth in SEQ ID NOs: 17, 18 and 19, respectively, and (b) a heavy chain variable region comprising heavy chain CDR1, CDR2 and CDR3 as set forth in SEQ ID NOs: 20, 21 and 22, respectively.

In other preferred embodiments of the treatment methods, medicaments and uses of the present invention, the VEGFR-2 antagonist is a monoclonal antibody, or antigen binding fragment thereof, which specifically binds to human VEGFR-2 and comprises (a) a heavy chain variable region comprising SEQ ID NO: 16 or a variant thereof, and (b) a light chain variable region comprising SEQ ID NO: 15 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

In another preferred embodiment of the treatment methods, medicaments and uses of the present invention, the VEGFR-2 antagonist is a monoclonal antibody that specifically binds to human VEGFR-2 and comprises (a) a heavy chain comprising SEQ ID NO: 14 and (b) a light chain comprising SEQ ID NO: 13 The heavy chain comprising SEQ ID NO: 14 comprises an IgG1 constant region. In another preferred embodiment of the treatment methods, medicaments and uses of the present invention, the VEGFR-2 antagonist is a monoclonal antibody that specifically binds to human VEGFR-2 and comprises (a) a heavy chain variable region comprising SEQ ID NO: 16 and (b) a light chain variable region comprising SEQ ID NO: 15.

Other examples of mAbs that bind to human VEGFR-2 and are useful in the treatment methods, medicaments and uses of the present invention are ramucirumab, also known as LY3009806, IMC-1121B, and Cyramza® (Eli Lilly & Co.) and related mAbs disclosed in PCT International application publ. No. WO2003075840A2.

In one embodiment, each of the anti-PD-1 and/or anti-VEGFR-2 antibodies or respective antigen-binding fragments thereof comprises a heavy chain constant region, e.g. a human constant region, such as □1, □2, □3, or □4 human heavy chain constant region or a variant thereof. In another embodiment, the anti-VEGFR-2 antibody or antigen-binding fragment thereof comprises a light chain constant region, e.g. a human light chain constant region, such as lambda (λ) or kappa (κ) human light chain region or a variant thereof. By way of example, and not limitation, the human heavy chain constant region can be □4 and the human light chain constant region can be kappa. In an alternative embodiment, the Fc region of the antibody is □4 with a Ser228Pro mutation (Schuurman, J et. al., Mol. Immunol. 38: 1-8, 2001).

In some embodiments, different constant domains may be appended to humanized VL and VH regions derived from the CDRs provided herein. For example, if a particular intended use of an antibody (or antigen binding fragment thereof) were to call for altered effector functions, a heavy chain constant domain other than human IgG1 may be used, or hybrid IgG1/IgG4 may be utilized.

Although human IgG1 antibodies provide for long half-life and for effector functions, such as complement activation and antibody-dependent cellular cytotoxicity, such activities may not be desirable for all uses of the antibody. In such instances, a human IgG4 constant domain, for example, may be used. The present invention includes the use of a combination of an anti-PD-1 antibody and an anti-VEGFR-2 (or a respective antigen-binding fragment thereof) antibody and wherein the anti-PD-1 antibody comprises an IgG4 constant domain and the anti-VEGFR-2 antibody comprises an IgG1 constant domain. In one embodiment, the IgG4 constant domain can differ from the native human IgG4 constant domain (Swiss-Prot Accession No. P01861.1) at a position corresponding to position 228 in the EU system and position 241 in the KABAT system, where the native Ser108 is replaced with Pro, to prevent a potential inter-chain disulfide bond between Cys106 and Cys109 (corresponding to positions Cys 226 and Cys 229 in the EU system and positions Cys 239 and Cys 242 in the KABAT system) that could interfere with proper intra-chain disulfide bond formation. See Angal et al. (1993) Mol. Imunol. 30:105. In other instances, a modified IgG1 constant domain which has been modified to increase half-life or reduce effector function can be used.

### Methods, Uses and Medicaments

A method for treating cancer in an individual is provided comprising co-administering to the individual a PD-1 antagonist and a VEGFR-2 antagonist. In another aspect of the invention, a method is provided for treating cancer in an individual comprising administering to the individual a composition comprising a PD-1 antagonist and a VEGFR-2 antagonist.

In another embodiment, a medicament is provided comprising a PD-1 antagonist for use in combination with a VEGFR-2 antagonist for treating a cancer. In yet another embodiment, a medicament is provided comprising a VEGFR-2 antagonist for use in combination with a PD-1 antagonist for treating cancer.

Other embodiments provide use of a PD-1 antagonist in the manufacture of a medicament for treating cancer in an individual when administered in combination with a VEGFR-2 antagonist and use of a VEGFR-2 antagonist in the manufacture of a medicament for treating a cancer in an individual when administered in combination with a PD-1 antagonist.

In another embodiment, the invention provides a VEGFR-2 antagonist for use in the treatment of cancer in an individual, wherein said use is in combination with a PD-1 antagonist. In a further embodiment, the invention provides a combination of a PD-1 antagonist and a VEGFR-2 antagonist for use in treatment of a subject with cancer.

In a still further embodiment, the invention provides use of a PD-1 antagonist and a VEGFR-2 antagonist in the manufacture of a medicament for treating cancer in an individual. In some embodiments, the medicaments comprise a kit, and the kit also comprises a package insert comprising instructions for using the PD-1 antagonist in combination with the VEGFR-2 antagonist to treat cancer in an individual.

In the foregoing methods, medicaments and uses, in one embodiment, the PD-1 antagonist and the VEGFR-2 antagonist are co-formulated. In another embodiment, the PD-1 antagonist and the VEGFR-2 antagonist are co-administered.

Cancers that may be treated by the combined compositions and methods of the invention include, but are not limited to: Cardiac cancers: sarcoma (angiosarcoma, fibrosarcoma, rhabdomyosarcoma, liposarcoma), myxoma, rhabdomyoma, fibroma, lipoma and teratoma; Lung cancers: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal cancers: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma) colorectal; Genitourinary tract cancers: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver cancers: hepatoma (hepatocellular carcinoma), cholangiocarcinoma, hepatoblastoma, angiosarcoma, hepatocellular adenoma, hemangioma; Bone cancers: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system cancers: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological cancer: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma)), fallopian tubes (carcinoma), Breast cancer; Hematologic cancers: blood (myeloid leukemia (acute and chronic), acute lymphoblastic leukemia, chronic lymphocytic leukemia, myeloproliferative diseases, multiple myeloma, myelodysplastic syndrome); hematopoietic tumors of the lymphoid lineage, including leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma; hematopoetic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma, and schwannomas; and other tumors, including melanoma, skin (non-melanomal) cancer, mesothelioma (cells), seminoma, teratocarcinoma, osteosarcoma, xenoderoma pigmentosum, keratoctanthoma, thyroid follicular cancer and Kaposi's sarcoma. In one embodiment, the forgoing cancers are advanced, unresectable or metastatic. In one embodiment, the patients are refractory to anti-PD-1 or anti-PD-L1 therapy.

In one embodiment, cancers that may be treated by the combined compositions and methods of the invention include, but are not limited to: a lung cancer, a pancreatic cancer, a colon cancer, a colorectal cancer, a myeloid leukemia, an acute myelogenous leukemia (AML), a chronic myelogenous leukemia (CML), a chronic myelomonocytic leukemia, a thyroid cancer, a myelodysplastic syndrome, a bladder carcinoma, an epidermal carcinoma, a melanoma, a breast cancer, a prostate cancer, a head and neck cancer, an ovarian cancer, a brain cancer, a cancer of mesenchymal origin, a sarcoma, a tetracarcinoma, a neuroblastoma, a kidney carcinoma, a hepatoma, a non-Hodgkin's lymphoma, a multiple myeloma, and an anaplastic thyroid carcinoma. In one embodiment, the cancer that may be treated is a cancer selected from the group consisting of melanoma, non-small cell lung cancer, head and neck squamous cell cancer, classical Hodgkin lymphoma, primary mediastinal B-cell lymphoma, urothelial carcinoma, microsatellite instability high (MSI-H) or a mismatch repair deficient (dMMR) solid tumor, a gastric cancer, squamous cell cancer of the esophagus, cervical cancer, hepatocellular carcinoma, Merkel cell carcinoma (MCC), and renal cell carcinoma (RCC). In another embodiment, the cancer is renal cell carcinoma (RCC), or a gastrointestinal stromal tumor. In a further embodiment, the cancer is adenoid cystic carcinoma or recurrent glioblastoma multiforme. In one embodiment, the forgoing cancers are advanced, unresectable, and/or metastatic. The patient to be treated with the drug combination can be refractory to anti-PD-1 or anti-PD-L1 therapy.

In one embodiment, cancers that may be treated by the combined compositions and methods of the invention include, but are not limited to: breast cancer, triple negative breast cancer (TNBC), metastatic breast cancer, and metastatic triple negative breast cancer (mTNBC). In one embodiment, cancers that may be treated by the antibodies, compositions and methods of the invention include glioblastoma multiforme (GBM) and recurrent glioblastoma multiforme (rGBM).

The combination therapy may also comprise one or more additional therapeutic agents. The additional therapeutic agent may be, e.g., a chemotherapeutic, a biotherapeutic agent, an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF). The specific dosage and dosage schedule of the additional therapeutic agent can further vary, and the optimal dose, dosing schedule and route of administration will be determined based upon the specific therapeutic agent that is being used.

Each therapeutic agent in a combination therapy of the invention may be administered either alone or in a medicament (also referred to herein as a pharmaceutical composition) that comprises the therapeutic agent and one or more pharmaceutically acceptable carriers, excipients and diluents, according to standard pharmaceutical practice.

Each therapeutic agent in a combination therapy of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some embodiments, the VEGFR-2 antagonist is administered before administration of the PD-1 antagonist, while in other embodiments, the VEGFR-2 antagonist is administered after administration of the PD-1 antagonist. The VEGFR-2 antagonist also can be administered concurrently with the PD-1 antagonist.

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

Each small molecule therapeutic agent in a combination therapy of the invention can be administered orally or parenterally, including the intravenous, intramuscular, intraperitoneal, subcutaneous, rectal, topical, and transdermal routes of administration.

A combination therapy of the invention may be used prior to or following surgery to remove or debulk a tumor and may be used prior to, during, or after radiation therapy.

In some embodiments, a combination therapy of the invention is administered to a patient who has not been previously treated with a biotherapeutic or chemotherapeutic agent, i.e., is treatment-naïve. In other embodiments, the combination therapy is administered to a patient who failed to achieve a sustained response after prior therapy with a biotherapeutic or chemotherapeutic agent, i.e., is treatment-experienced.

A combination therapy of the invention is typically used to treat a tumor that is large enough to be found by palpation or by imaging techniques well known in the art, such as MRI, ultrasound, or CAT scan.

Selecting a dosage regimen (also referred to herein as an administration regimen) for a combination therapy of the invention depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of each therapeutic agent delivered to the patient consistent with an acceptable level of side effects. Accordingly, the dose amount and dosing frequency of each biotherapeutic and chemotherapeutic agent in the combination depends in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348: 601-608; Milgrom et al. (1999) New Engl. J. Med. 341: 1966-1973; Slamon et al. (2001) New Engl. J. Med. 344: 783-792; Beniaminovitz et al. (2000) New Engl. J. Med. 342: 613-619; Ghosh et al. (2003) New Engl. J. Med. 348: 24-32; Lipsky et al. (2000) New Engl. J. Med. 343: 1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Biotherapeutic agents in a combination therapy of the invention may be administered by continuous infusion, or by doses at intervals of, e.g., daily, every other day, three times per week, or one time each week, two weeks, three weeks, monthly, bimonthly, etc. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349: 427-434; Herold et al. (2002) New Engl. J. Med. 346:1692-1698; Liu et al. (1999) J. Neurol. Neurosurg. Psych. 67: 451-456; Portielji et al. (2003) Cancer Immunol. Immunother. 52: 133-144.

In a preferred embodiment of the invention, VEGFR-2 antagonist in the combination therapy is olinvacimab or an olinvacimab variant, which may be administered from 2 to 24 mg/kg, weekly, every two weeks, or every three weeks, depending on tumor type, and patient factors.

In some embodiments that employ an anti-human PD-1 mAb as the PD-1 antagonist in the combination therapy, the dosing regimen will comprise administering the anti-human PD-1 mAb at a dose of 1, 2, 3, 5 or 10 mg/kg at intervals of about 14 days (±2 days) or about 21 days (+2 days) or about 30 days (±2 days) throughout the course of treatment.

In other embodiments that employ an anti-human PD-1 mAb as the PD-1 antagonist in the combination therapy, the dosing regimen will comprise administering the anti-human PD-1 mAb at a dose of from about 0.005 mg/kg to about 10 mg/kg, with intra-patient dose escalation. In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (±2 days), for doses subsequent to the second dose.

In certain embodiments, a subject receiving the combination therapy will be administered an intravenous (IV) infusion or subcutaneous injection of a medicament comprising any of the PD-1 antagonists described herein.

In one preferred embodiment of the invention, the PD-1 antagonist in the combination therapy is nivolumab, which is administered intravenously at a dose selected from the group consisting of: 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg/kg Q3W.

In another preferred embodiment of the invention, the PD-1 antagonist in the combination therapy is pembrolizumab, or a pembrolizumab variant, which is administered in a liquid medicament at a dose selected from the group consisting of 1 mg/kg Q2W, 2 mg/kg Q2W, 3 mg/kg Q2W, 5 mg/kg Q2W, 10 mg/kg Q2W, 1 mg/kg Q3W, 2 mg/kg Q3W, 3 mg/kg Q3W, 5 mg/kg Q3W, 10 mg/kg Q3W and flat-dose equivalents of any of these doses, i.e., such as 200 mg Q3W. In some embodiments, pembrolizumab is provided as a liquid medicament that comprises 25 mg/ml pembrolizumab, 7% (w/v) sucrose, 0.02% (w/v) polysorbate 80 in 10 mM histidine buffer pH 5.5. In other embodiments, pembrolizumab is provided as a liquid medicament that comprises about 125 to about 200 mg/mL of pembrolizumab, or an antigen binding fragment thereof; about 10 mM histidine buffer; about 10 mM L-methionine, or a pharmaceutically acceptable salt thereof; about 7% (w/v) sucrose; and about 0.02% (w/v) polysorbate 80.

In some embodiments, the selected dose of pembrolizumab is administered by IV infusion. In one embodiment, the selected dose of pembrolizumab is administered by IV infusion over a time period of between 25 and 40 minutes, or about 30 minutes.

In some embodiments, the patient is treated with the combination therapy for at least 24 weeks, e.g., eight 3-week cycles. In some embodiments, treatment with the combination therapy continues until the patient exhibits evidence of PD or a CR.

Pembrolizumab may be administered 200 mg every 3 weeks or 400 mg every 6 weeks, depending on tumor type, and patient factors. Pembrolizumab may be administered at 200 mg intravenously every three weeks starting on day 1 of a 21 day cycle. The route of administration may be varied in any way, limited by the physical properties of the drugs and the convenience of the patient.

Olinvacimab may be used in simultaneous, separate, or sequential combination with pembrolizumab in the treatment of cancer, for example a metastatic triple-negative breast cancer (TNBC). Olinvacimab can be administered at a dose of 8 mg/kg to 16 mg/kg every week, for example, 8 mg/kg, 9 mg/kg, 10 mg/kg, 11 mg/kg, 12 mg/kg, 13 mg/kg, 14 mg/kg, 15 mg/kg, or 16 mg/kg, or at a dose of 12 mg/kg to 24 mg/kg every 2 weeks, for example, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, or 24 mg/kg, and pembrolizumab is administered at a dose of 200 mg every 3 weeks. Olinvacimab can be administered at a dose of 8 mg/kg to 20 mg/kg every week, for example, 8 mg/kg, 9 mg/kg, 10 mg/kg, 11 mg/kg, 12 mg/kg, 13 mg/kg, 14 mg/kg, 15 mg/kg, 16 mg/kg, 18, mg/kg, or 20 mg/kg, or at a dose of 12 mg/kg to 24 mg/kg every 2 weeks, for example, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, or 24 mg/kg, and pembrolizumab is administered at a dose of 400 mg every 6 weeks.

In the foregoing methods, medicaments and uses, in another embodiment, the anti-PD-1 or anti-PD-L1 antibody and anti-VEGFR-2 antibody are co-administered. In one embodiment, 200 mg pembrolizumab or a pembrolizumab variant is administered by IV infusion on Day 1 every three weeks, and 12 mg/kg olinvacimab or an olinvacimab variant is administered by IV infusion on Day 1 every week. In another embodiment, 200 mg pembrolizumab or a pembrolizumab variant is administered by IV infusion on Day 1 every three weeks, and 16 mg/kg olinvacimab or an olinvacimab variant is administered by IV infusion on Day 1 every week. In another embodiment, 200 mg pembrolizumab or a pembrolizumab variant is administered by IV infusion on Day 1 every three weeks, and 18 mg/kg or 20 mg/kg olinvacimab or an olinvacimab variant is administered by IV infusion on Day 1 every week.

In the foregoing methods, medicaments and uses, in one embodiment, 400 mg pembrolizumab or a pembrolizumab variant is administered on Day 1 every six weeks, and 12 mg/kg olinvacimab or an olinvacimab variant is administered by IV infusion on Day 1 every week for intravenous infusion. In another embodiment, 400 mg pembrolizumab or a pembrolizumab variant is administered on Day 1 every six weeks, and 16 mg/kg olinvacimab or an olinvacimab variant is administered by IV infusion on Day 1 every week for intravenous infusion.

The VEGFR-2 antagonist and PD-1 antagonist can be respectively administered in dosage forms, for example without limitation, reconstitutable powders, elixirs, liquids, solutions, suspensions, emulsions, powders, granules, particles, microparticles, dispersible granules, cachets, inhalants, aerosol inhalants, patches, particle inhalants, implants, depot implants, injectables (including subcutaneous, intramuscular, intravenous, and intradermal), infusions, and combinations thereof.

In one embodiment, the anti-VEGFR-2 antagonist and PD-1 antagonist are respectively administered as an intravenous (IV) infusion. In another embodiment, the anti-VEGFR-2 antagonist and PD-1 antagonist are respectively administered as a subcutaneous injection.

The present invention also provides a medicament that comprises a PD-1 or VEGFR-2 antagonist as described above and a pharmaceutically acceptable excipient. When the PD-1 antagonist or VEGFR-2 antagonist is a biotherapeutic agent, e.g., a mAb, the antagonist may be produced in CHO cells using conventional cell culture and recovery/purification technologies.

Pharmaceutically acceptable excipients of the present disclosure include for instance, solvents, bulking agents, buffering agents, tonicity adjusting agents, and preservatives (see, e.g., Pramanick et al., Pharma Times, 45: 65-77, 2013). In some embodiments the pharmaceutical compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent). The pharmaceutical compositions of the present disclosure are suitable for parenteral administration.

In some embodiments, the pharmaceutical compositions comprise an aqueous vehicle as a solvent. Suitable vehicles include for instance sterile water, saline solution, phosphate buffered saline, and Ringer's solution. In some embodiments, the composition is isotonic.

The pharmaceutical compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose, and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 4 to 9. In some embodiments, the pH is greater than (lower limit) 4, 5, 6, 7 or 8. In some embodiments, the pH is less than (upper limit) 9, 8, 7, 6 or 5. That is, the pH is in the range of from about 4 to 9 in which the lower limit is less than the upper limit.

The pharmaceutical compositions may comprise a tonicity adjusting agent. Suitable tonicity adjusting agents include for instance dextrose, glycerol, sodium chloride, glycerin, and mannitol.

The pharmaceutical compositions may comprise a preservative. Suitable preservatives include for instance antioxidants and antimicrobial agents. However, in preferred embodiments, the pharmaceutical composition is prepared under sterile conditions and is in a single use container, and thus does not necessitate inclusion of a preservative.

In some embodiments, a medicament comprising an anti-PD-1 antibody as the PD-1 antagonist may be provided as a liquid formulation or prepared by reconstituting a lyophilized powder with sterile water for injection prior to use. PCT International application publ. No. WO 2012/135408 describes the preparation of liquid and lyophilized medicaments comprising pembrolizumab that are suitable for use in the present invention. In some embodiments, a medicament comprising pembrolizumab is provided in a glass vial that contains about 100 mg of pembrolizumab in 4 ml of solution. Each 1 mL of solution contains 25 mg of pembrolizumab and is formulated in: L-histidine (1.55 mg), polysorbate 80 (0.2 mg), sucrose (70 mg), and Water for Injection, USP. The solution requires dilution for IV infusion.

The medicaments described herein may be provided as a kit that comprises a first container, a second container and a package insert or label. The first container contains at least one dose of a medicament comprising a PD-1 antagonist, the second container contains at least one dose of a medicament comprising a VEGFR-2 antagonist, and the package insert or label that comprises instructions for treating a patient for cancer using the medicaments. The first and second containers may be comprised of the same or different shapes (e.g., vials, syringes and bottles) and/or materials (e.g., plastic or glass). The kit may further comprise other materials that may be useful in administering the medicaments, such as diluents, filters, IV bags and lines, needles and syringes.

These and other aspects of the invention, including the exemplary specific embodiments listed below, will be apparent from the teachings contained herein.

### Exemplary Specific Embodiments of the Invention

1. A VEGFR-2 antagonist for use in the treatment of a cancer, wherein the use is in combination with a PD-1 antagonist.
2. The VEGFR-2 antagonist for use of embodiment 1, wherein the PD-1 antagonist is a monoclonal antibody, or an antigen binding fragment thereof.
3. The VEGFR-2 antagonist for use of embodiment 1, wherein the individual is a human and the PD-1 antagonist is a monoclonal antibody, or an antigen binding fragment thereof, which specifically binds to human PD-1 and blocks the binding of human PD-L1 to human PD-1.
4. The VEGFR-2 antagonist for use of embodiment 3, wherein the PD-1 antagonist also blocks binding of human PD-L2 to human PD-1
5. The VEGFR-2 antagonist for use of embodiment 4, wherein the PD-1 antagonist is a monoclonal antibody, or antigen binding fragment thereof, which comprises: (a) light chain CDR1, CDR2 and CDR3 of SEQ ID NOs: 1, 2 and 3, respectively, and (b) heavy chain CDR1, CDR2 and CDR3 of SEQ ID NOs: 6, 7 and 8, respectively.
6. The VEGFR-2 antagonist for use of embodiment 4, wherein the PD-1 antagonist is an anti-PD-1 monoclonal antibody that comprises a heavy chain and a light chain, and wherein the heavy chain comprises a heavy chain variable region comprising SEQ ID NO: 9 and the light chain comprises a light chain variable region comprising SEQ ID NO: 4.
7. The VEGFR-2 antagonist for use of embodiment 4, wherein the PD-1 antagonist is an anti-PD-1 monoclonal antibody that comprises a heavy chain and a light chain, and wherein the heavy chain comprises SEQ ID NO: 10 and the light chain comprises SEQ ID NO: 5.
8. The VEGFR-2 antagonist for use of embodiment 4, wherein the PD-1 antagonist is pembrolizumab.
9. The VEGFR-2 antagonist for use of embodiment 4, wherein the PD-1 antagonist is a pembrolizumab variant.
10. The VEGFR-2 antagonist for use of embodiment 4, wherein the PD-1 antagonist is nivolumab.
11. The VEGFR-2 antagonist for use of any one of embodiments 1 to 10, wherein the VEGFR-2 antagonist is a monoclonal antibody, or an antigen binding fragment thereof, that blocks the binding of VEGFR-2 to VEGF.
12. The VEGFR-2 antagonist for use of any one of embodiments 1 to 10, wherein the VEGFR-2 antagonist is an antibody, or antigen binding fragment thereof, which comprises: (a) light chain CDR1, CDR2 and CDR3 of SEQ ID NOs: 17, 18 and 19, respectively, and (b) heavy chain CDR1, CDR2 and CDR3 of SEQ ID NOs: 20, 21 and 22, respectively.
13. The VEGFR-2 antagonist for use of any one of embodiments 1 to 10, wherein the VEGFR-2 antagonist is an anti-VEGFR-2 antibody that comprises a heavy chain and a light chain, and wherein the heavy chain comprises a heavy chain variable region comprising SEQ ID NO: 16 and the light chain comprises a light chain variable region comprising SEQ ID NO: 15.
14. The VEGFR-2 antagonist for use of any one of embodiments 1 to 10, wherein the VEGFR-2 antagonist is an anti-VEGFR-2 antibody that comprises a heavy chain and a light chain, and wherein the heavy chain comprises SEQ ID NO: 14 and the light chain comprises SEQ ID NO: 13.
15. The VEGFR-2 antagonist for use of any one of embodiments 1 to 10, wherein the VEGFR-2 antagonist is an olinvacimab variant.
16. The VEGFR-2 antagonist for use of any one of embodiments 1 to 10, wherein the VEGFR-2 antagonist is ramucirumab.
17. The VEGFR-2 antagonist for use of embodiment 1, wherein the PD-1 antagonist is a humanized anti-PD-1 antibody that comprises a heavy chain and a light chain, and wherein the heavy chain comprises a heavy chain variable region comprising heavy chain CDR1, CDR2 and CDR3 of SEQ ID NOs: 6, 7 and 8, respectively, and the light chain comprises a light chain variable region comprising light chain CDR1, CDR2 and CDR3 of SEQ ID NOs: 1, 2 and 3, respectively; and the VEGFR-2 antagonist is a humanized anti-VEGFR-2 antibody that comprises a heavy chain and a light chain, and wherein the heavy chain comprises a heavy chain variable region comprising heavy chain CDR1, CDR2 and CDR3 of SEQ ID NOs: 20, 21 and 22, respectively, and the light chain comprises a light chain variable region comprising light chain CDR1, CDR2 and CDR3 of SEQ ID NOs: 17, 18 and 19, respectively.
18. The VEGFR-2 antagonist for use of embodiment 1, wherein the PD-1 antagonist is an anti-PD-1 antibody that comprises a heavy chain and a light chain, and wherein the heavy chain comprises a heavy chain variable region comprising SEQ ID NO: 9 and the light chain comprises a light chain variable region comprising SEQ ID NO: 4; and the VEGFR-2 antagonist is an anti-VEGFR-2 antibody that comprises a heavy chain and a light chain, and wherein the heavy chain comprises a heavy chain variable region comprising SEQ ID NO: 16 and the light chain comprises a light chain variable region comprising SEQ ID NO: 15.
19. The VEGFR-2 antagonist for use of embodiment 1, wherein the PD-1 antagonist is an anti-PD-1 antibody that comprises a heavy chain and a light chain, and wherein the heavy chain comprises SEQ ID NO: 10 and the light chain comprises SEQ ID NO: 5; and the VEGFR-2 antagonist is an anti-VEGFR-2 antibody that comprises a heavy chain and a light chain, and wherein the heavy chain comprises SEQ ID NO: 14 and the light chain comprises SEQ ID NO: 13.
20. The VEGFR-2 antagonist for use of any one of embodiments 1 to 19, wherein the PD-1 antagonist and VEGFR-2 antagonist are co-formulated.
21. The VEGFR-2 antagonist for use of any one of embodiments 1 to 19, wherein the PD-1 antagonist and VEGFR-2 antagonist are co-administered.
22. The VEGFR-2 antagonist for use of any one of embodiments 1 to 21, wherein the individual has not been previously treated with anti-PD-1 or anti-PD-L1 therapy or is confirmed progressive while receiving prior anti-PD-1 therapy.
23. The VEGFR-2 antagonist for use of any one of embodiments 1 to 22, wherein the cancer is a breast cancer, a glioblastoma or a metastatic cancer.
24. The VEGFR-2 antagonist for use of any one of embodiments 1 to 22, wherein the cancer is triple negative breast cancer.
25. The VEGFR-2 antagonist for use of any one of embodiments 1 to 22, wherein the cancer is metastatic triple negative breast cancer.
26. The VEGFR-2 antagonist for use of any one of embodiments 1 to 25, wherein 200 mg pembrolizumab or a pembrolizumab variant is administered by IV infusion on Day 1 every three weeks, and 16 mg/kg olinvacimab or an olinvacimab variant is administered by IV infusion on Day 1 every week.
27. The VEGFR-2 antagonist for use of any one of embodiments 1 to 25, wherein 400 mg pembrolizumab or a pembrolizumab variant is administered on Day 1 every six weeks, and 16 mg/kg olinvacimab or an olinvacimab variant is administered by IV infusion on Day 1 every week for intravenous infusion.
28. The VEGFR-2 antagonist for use of any one of embodiments 26 to 27, wherein the cancer is triple-negative breast cancer.
29. The VEGFR-2 antagonist for use of any one of embodiments 26 to 27, wherein the cancer is metastatic triple-negative breast cancer.
30. A method of treating a cancer in a patient comprising administering an anti-VEGFR-2 antagonist and PD-1 antagonist to the patient.
31. The method of embodiment 30, wherein the VEGFR2 antagonist is olinvacimab and the PD-1 antagonist is pembrolizumab.
32. The method of embodiment 30 or 31, wherein the cancer comprises a breast cancer, preferably metastatic triple-negative breast cancer.
33. The method of embodiment 31, wherein the olinvacimab is administered at a dose from about 8 to 16 mg per kg once weekly or 12 to 24 mg per kg once every 2 weeks in combination with pembrolizumab administered at a dose of 200 mg once every 3 weeks or at a dose of 400 mg every 6 weeks.
34. The method of any one of embodiments 30 to 33, wherein 200 mg pembrolizumab or a pembrolizumab variant is administered by IV infusion on Day 1 every three weeks, and 16 mg/kg olinvacimab or an olinvacimab variant is administered by IV infusion on Day 1 every week.
35. The method of any one of embodiments 30 to 33, wherein 400 mg pembrolizumab or a pembrolizumab variant is administered on Day 1 every six weeks, and 16 mg/kg olinvacimab or an olinvacimab variant is administered by IV infusion on Day 1 every week for intravenous infusion.
36. The method of any one of embodiments 34 to 35, wherein the cancer is triple-negative breast cancer.
37. The method of any one of embodiments 34 to 35, wherein the cancer is metastatic triple-negative breast cancer.
38. A composition for use in a manufacture of a medicament for combined therapy method of treating a cancer in a patient comprising a VEGFR-2 antagonist and a PD-1 antagonist.
39. The composition of embodiment 38, wherein the VEGFR-2 antagonist is an anti-VEGFR-2 antibody.
40. The composition of embodiment 38 or 39, wherein the PD-1 antagonist is an anti-PD-1 antibody.
41. The composition of embodiment 38 or 39, wherein the cancer is a breast cancer, a glioblastoma, or a metastatic cancer.
42. The composition of embodiment 38 or 39, wherein the breast cancer comprises triple-negative breast cancer.
43. The composition of embodiment 38 or 39, wherein the cancer comprises metastatic triple-negative breast cancer.
44. A composition for use in a manufacture of a medicament for combined therapy method of treating a metastatic triple-negative breast cancer in a patient having a lung or brain metastatic lesion, comprising a VEGFR-2 antagonist and PD-1 antagonist.
45. The composition of embodiment 44, wherein the VEGFR-2 antagonist is an anti-VEGFR-2 antibody.
46. The composition of embodiment 44 or 45, wherein the PD-1 antagonist is an anti-PD-1 antibody.
47. A combined therapy method of treating a cancer in a patient comprising administering to the patient a therapeutically effective amount of a VEGFR-2 antagonist and a PD-1 antagonist.
48. A combined therapy method of treating a cancer in a patient having a lung or a brain metastatic lesion, comprising administering to the patient a therapeutically effective amount of a VEGFR-2 antagonist and a PD-1 antagonist.
49. The combined therapy method of embodiment 47 or 48, wherein the VEGFR-2 antagonist is an anti-VEGFR antibody.
50. The combined therapy method of any of embodiments 47 to 49, wherein the PD-1 antagonist is an anti-PD-1 antibody.
51. The combined therapy method of any of embodiments 47 to 50, wherein the cancer comprises a breast cancer, preferably metastatic triple-negative breast cancer.
52. A combined therapy method of treating a cancer in a patient comprising administering to the patient a therapeutically effective amount of an anti-VEGFR-2 antibody and an anti-PD-1 antibody.
53. A combined therapy method of treating a cancer in a patient having a lung or a brain metastatic lesion, comprising administering to the patient a therapeutically effective amount of an anti-VEGFR-2 antibody and an anti-PD-1 antibody.
54. Anti-VEGFR2 for use in any embodiment of this disclosure can be selected from the group including, but not limited to, olinvacimab and ramucirumab.
55. Anti-PD-1 antibody for use in any embodiment of this disclosure can be selected from the group including, but not limited to, pembrolizumab, nivolumab, cemiplimab, camrelizumab, sintilimab, tislelizumab, and toripalimab.
56. A combination therapy of olinvacimab and pembrolizumab to treat a triple-negative breast cancer having a lung or brain metastatic lesion (lung or brain metastatic mTNBC).
57. A method of treating a cancer in a patient comprising administering an anti-VEGFR-2 antagonist and PD-1 antagonist to the patient.
58. The method of embodiment 57, wherein the VEGFR2 antagonist is olinvacimab and the PD-1 antagonist is pembrolizumab.
59. The method of embodiment 57 or 58, wherein the cancer is a breast cancer, preferably metastatic triple-negative breast cancer.
60. The method of embodiment 58, wherein the olinvacimab is administered at a dose from about 8 to 16 mg per kg once weekly or 12 to 24 mg per kg once every 2 weeks in combination with pembrolizumab administered at a dose of 200 mg every 3 weeks or at a dose of 400 mg every 6 weeks.

### General Methods

Standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif.). Standard methods also appear in Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., N.Y.). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165: 6205; He, et al. (1998) J. Immunol. 160:1029; Tang et al. (1999) J. Biol. Chem. 274: 27371-27378; Baca et al. (1997) J. Biol. Chem. 272: 10678-10684; Chothia et al. (1989) Nature 342: 877-883; Foote and Winter (1992) J. Mol. Biol. 224: 487-499; U.S. Pat. No. 6,329,511).

An alternative to humanization is to use human antibody libraries displayed on phage or human antibody libraries in transgenic mice (Vaughan et al. (1996) Nature Biotechnol. 14: 309-314; Barbas (1995) Nature Medicine 1:837-839; Mendez et al. (1997) Nature Genetics 15: 146-156; Hoogenboom and Chames (2000) Immunol. Today 21: 371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17: 397-399).

Purification of antigen is not necessary for the generation of antibodies. Animals can be immunized with cells bearing the antigen of interest. Splenocytes can then be isolated from the immunized animals, and the splenocytes can fuse with a myeloma cell line to produce a hybridoma (see, e.g., Meyaard et al. (1997) Immunity 7: 283-290; Wright et al. (2000) Immunity 13: 233-242; Preston et al., supra; Kaithamana et al. (1999) J. Immunol. 163: 5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146: 169-175; Gibellini et al. (1998) J. Immunol. 160: 3891-3898; Hsing and Bishop (1999) J. Immunol. 162: 2804-2811; Everts et al. (2002) J. Immunol. 168: 883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, PA; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68: 177-181; von Heijne (1983) Eur. J. Biochem. 133: 17-21; von Heijne (1986) Nucleic Acids Res. 14: 4683-4690).

### EXAMPLES

### Design of Clinical Trials

Phase 1b, open-label, safety and tolerability studies were designed of olinvacimab in combination with pembrolizumab in patients with metastatic triple-negative breast cancer (mTNBC) or patients with recurrent glioblastoma multiforme (rGBM).

The primary endpoint of these trials was to determine the safety and tolerability of the drug combination and to establish a preliminary recommended Phase 2 dose (RP2D) of olinvacimab administered in combination with pembrolizumab in patients with mTNBC or rGBM.

rGBM

Nine (9) patients with recurrent glioblastoma multiforme were enrolled into this trial and treated with olinvacimab 12 mg/kg or 16 mg/kg weekly (q7d) infusion in combination with pembrolizumab 200 mg day 1 in 3 week (q21d) cycles.

mTNBC

Eleven (11) patients with metastatic triple-negative breast cancer (ER, PR & HER2-negative MBC with at least one measurable lesion) were enrolled into this trial and treated with olinvacimab 12 mg/kg or 16 mg/kg weekly (q7d) infusion in combination with pembrolizumab 200 mg day 1 in 3 week (q21d) cycles. The patients ranged in age from 39 to 67. All 11 patients tested were female. Five (5) patients had previous chemotherapy (anthracycline and taxanes) for mTNBC (with 3 patients also having received immunotherapy). Six (6) patients were treated in the first-line metastatic setting. For the 11 patients, metastases were found in the lung (7 patients), lymph node (6 patients), bone (3 patients), liver (3 patients), brain (3 patients), and in other sites for 5 patients (adrenal nodules, skin, kidney, chest wall and thoracic).

In addition, efficacy endpoints such as ORR, DCR, OS and PFS, were evaluated by tumor assessment done at the end of every 2nd cycle of administering drug and/or study termination visit. RECIST 1.1 criteria was used to evaluate efficacy.

### Result of Clinical Trials

rGBM

Three (3) patients received olinvacimab at 12 mg/kg with pembrolizumab, completing a median of 3 cycles (range 2-6). Six (6) patients were treated with olinvacimab at 16 mg/kg with pembrolizumab, completing a median of 3 cycles (range 2-12).

The interim result is that four (4) patients (44%) had stable disease (SD) as a best response. One patient had SD for over 12 cycles (currently 15 cycles, 10 months). Treatment was ceased due to disease progression (PD) in eight (8) patients. The median overall survival (OS) was 7.2 months (range 2.1 to 14.6 months). Median progression free survival (PFS) was 1.3 months (range 1.2 to 8.3 months).

mTNBC

Five (5) patients received olinvacimab at 12 mg/kg with pembrolizumab (Dose Level 1). Dose Level 1 refers to olinvacimab 12 mg/kg weekly (q7d) infusion in combination with pembrolizumab 200 mg day 1 in 3 week (q21d) cycles. The treatment cycles completed at Dose Level 1 were 1, 2, 12, 18, and 6 (range of 1-18). Thus, the 5 patients completed a median of 6 cycles (range 1-18). Six (6) patients were treated with olinvacimab at 16 mg/kg with pembrolizumab (Dose Level 2). Dose Level 2 refers to olinvacimab 16 mg/kg weekly (q7d) infusion in combination with pembrolizumab 200 mg day 1 in 3 week (q21d) cycles. The treatment cycles completed at Dose Level 2 were 2, 2, 8, 9, 14, and 21 (range 2-21). Thus, the 6 patients at Dose Level 2 completed a median of 8 cycles (range 2-21).

No dose limiting toxicity (DLT) was observed. All patients experienced treatment emergent adverse events (TEAEs), TEAEs≥grade 3 were seen in 6 patients (27 events), with 8 events being related to treatment which included pulmonary embolism, hypertension, arthralgia, thromboembolic event, myositis and hyponatremia. Eight serious adverse events (SAEs) occurred to 5 patients and included pulmonary embolism, disease progression, pain, myositis, seizure, hypotension and thromboembolic event. Table 5 provides a summary of treatment emergent adverse events in patients treated with olinvacimab and pembrolizumab.

TABLE 5
Treatment emergent adverse events

| | Dose Level 1 | Dose Level 2 | Overall |
|---|---|---|---|
| Subjects with at | (N = 5) | (N = 6) | (N = 11) |
| least one TEAE | 5 (100%) | 6 (100%) | 11 (100%) |
| Haemangioma of skin | 3 (60.0%) | 5 (83.3%) | 8 (72.7%) |
| Fatigue | 1 (20.0%) | 4 (66.7%) | 5 (45.5%) |
| Nausea | 2 (40.0%) | 3 (50.0%) | 5 (45.5%) |
| Dizziness | 1 (20.0%) | 3 (50.0%) | 4 (36.4%) |
| Headache | 2 (40.0%) | 2 (33.3%) | 4 (36.4%) |
| Rash | 4 (80.0%) | 0 | 4 (36.4%) |
| Oedema peripheral | 1 (20.0%) | 2 (33.3%) | 3 (27.3%) |
| Abdominal pain | 1 (20.0%) | 2 (33.3%) | 3 (27.3%) |
| Constipation | 2 (40.0%) | 1 (16.7%) | 3 (27.3%) |
| Diarrhea | 1 (20.0%) | 2 (33.3%) | 3 (27.3%) |
| Dyspepsia | 0 | 3 (50.0%) | 3 (27.3%) |
| Vomiting | 2 (40.0%) | 1 (16.7%) | 3 (27.3%) |
| Arthralgia | 1 (20.0%) | 2 (33.3%) | 3 (27.3%) |
| Myalgia | 2 (40.0%) | 1 (16.7%) | 3 (27.3%) |
| Cough | 1 (20.0%) | 2 (33.3%) | 3 (27.3%) |
| Dyspnea | 2 (40.0%) | 1 (16.7%) | 3 (27.3%) |
| Pollakiuria | 1 (20.0%) | 2 (33.3%) | 3 (27.3%) |

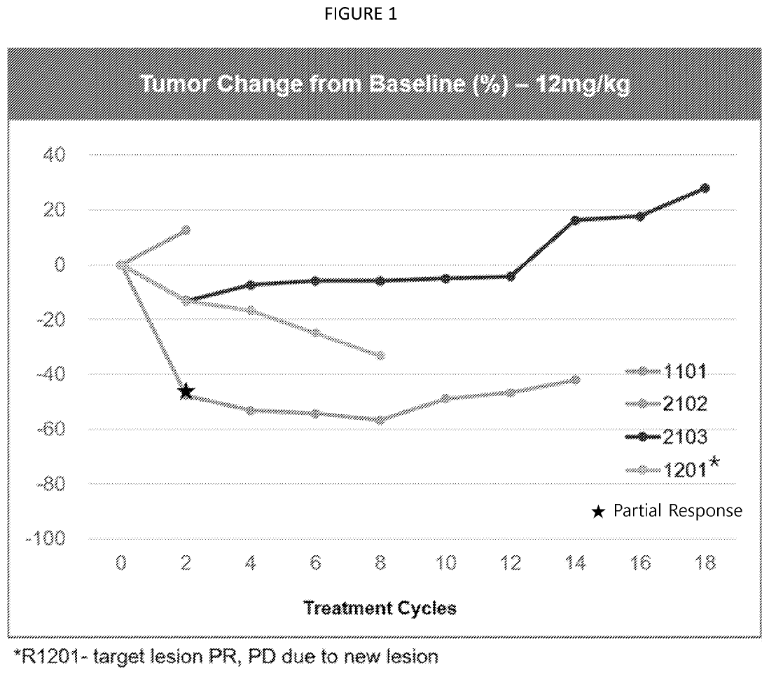

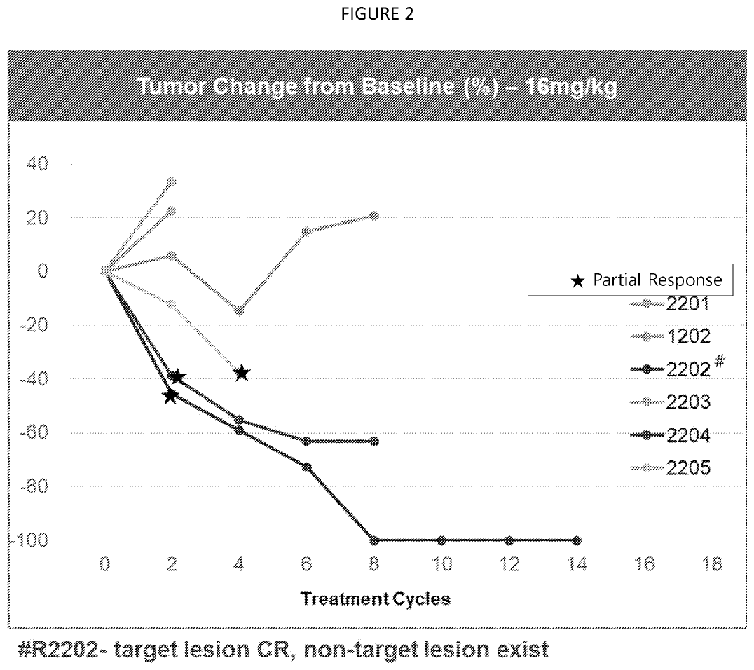

Tumor assessment was performed at the end of every 2nd cycle of drug administration and/or at a study termination visit. Tumor size change from baseline data for four patients treated with 12 mg/kg olinvacimab with pembrolizumab is depicted in FIG. 1. Tumor size change from baseline data for six patients treated with 16 mg/kg olinvacimab with pembrolizumab is depicted in FIG. 2.

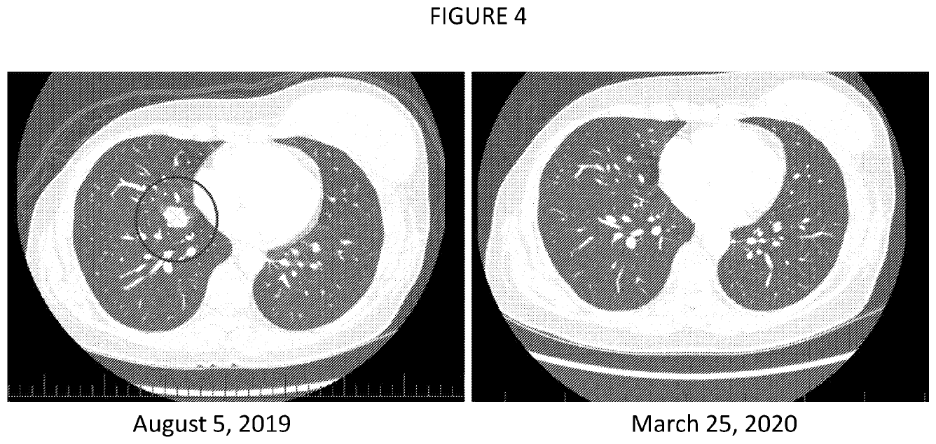

Four (4) patients (36%) had partial response (PR) as best overall response. See black asterisks in FIGS. 1 and 2. One patient had complete response (CR) in target lesion (see data for patient 2202 in FIG. 2 and FIG. 4). Due to a non-target lesion that remained, patient 2202 was evaluated as an overall partial response (PR). Five (5) patients had clinical benefit (PR+SD≥24 weeks). Median progression free survival (PFS) was 4.2 months (range 0.5 to 10.7 months) as of June 2020. Treatment was ceased due to disease progression (PD) in seven (7) patients. Four (4) patients received treatment at data cut-off.

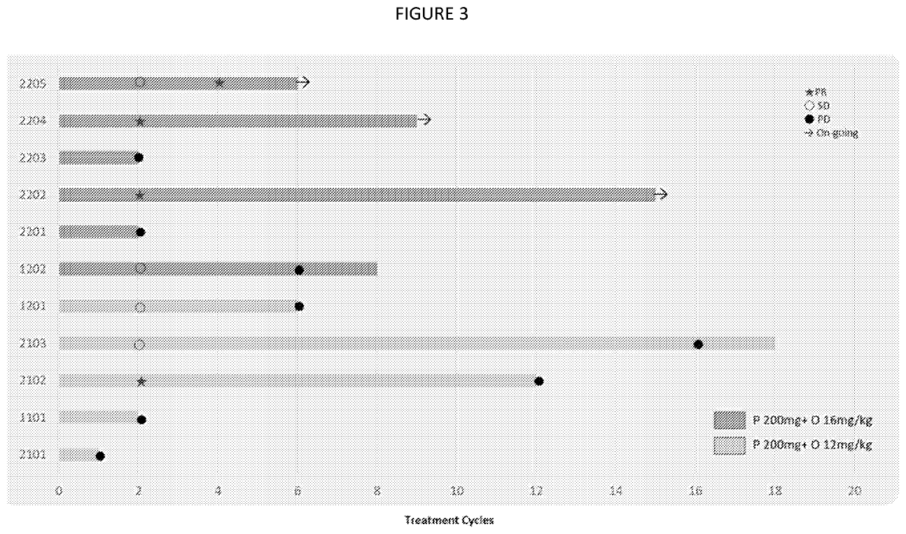

A summary of the interim result for the eleven patients in the clinical trial is depicted in FIG. 3.

Four (4) of the 11 patients who were enrolled in this Phase I clinical trial had lung metastatic lesions at the time they were enrolled. These lesions were measured and assessed according to RECIST 1.1 criteria. As a best response, two (2) patients of these 4 patients showed PR (partial response) and one (1) patient showed SD (stable disease) more than 36 weeks after treatment commenced. Patient 2202 showed disappearance of the target lesion in the lung (FIG. 4), indicating complete response of the target lesion. As noted above, due to the presence of a non-target lesion that remained, patient 2202 was evaluated as overall partial response (PR). This means a combination therapy of olinvacimab and pembrolizumab has pharmacologic effect to reduce the size of metastatic lung lesion in triple-negative breast cancer or inhibit deterioration of disease. The clinical data show on whole clear evidence of improved efficacy in treatment of mTNBC. In conclusion, combination therapy of olinvacimab and pembrolizumab was well tolerated in mTNBC patients with a clear evidence of clinical benefit observed especially in the cohort that was treated with olinvacimab 16 mg/kg. The improved efficacy may indicate that olinvacimab plays a pivotal role in tumor microenvironment (TME) both as an angiogenesis inhibitor and an immune-modulator.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

1. A method for treating a cancer in an individual comprising administering to the individual a PD-1 antagonist and a VEGFR-2 antagonist.

2. The method of claim 1, wherein the PD-1 antagonist is a monoclonal antibody, or an antigen binding fragment thereof.

3. The method of claim 1, wherein the individual is a human and the PD-1 antagonist is a monoclonal antibody, or an antigen binding fragment thereof, which specifically binds to human PD-1 and blocks the binding of human PD-L1 to human PD-1.

4. The method of claim 3, wherein the PD-1 antagonist also blocks binding of human PD-L2 to human PD-1.

5. The method of claim 4, wherein the PD-1 antagonist is an anti-PD-1 antibody, or antigen binding fragment thereof, which comprises: (a) a light chain variable region comprising light chain CDR1, CDR2, and CDR3 of SEQ ID NOs: 1, 2 and 3, respectively, and (b) a heavy chain variable region comprising heavy chain CDR1, CDR2 and CDR3 of SEQ ID NOs: 6, 7 and 8, respectively.

6. The method of claim 4, wherein the PD-1 antagonist is an anti-PD-1 antibody that comprises a heavy chain and a light chain, and wherein the heavy chain comprises a heavy chain variable region comprising SEQ ID NO: 9, and the light chain comprises a light chain variable region comprising SEQ ID NO: 4.

7. The method of claim 4, wherein the PD-1 antagonist is an anti-PD-1 antibody that comprises a heavy chain and a light chain, and wherein the heavy chain comprises SEQ ID NO: 10 and the light chain comprises SEQ ID NO: 5.

8. The method of claim 4, wherein the PD-1 antagonist is pembrolizumab.

9. The method of claim 4, wherein the PD-1 antagonist is a pembrolizumab variant.

10. The method of claim 4, wherein the PD-1 antagonist is nivolumab.

11. The method of any one of claims 1 to 10, wherein the VEGFR-2 antagonist is a monoclonal antibody, or an antigen binding fragment thereof, that blocks binding of VEGFR-2 to VEGF.

12. The method of any one of claims 1 to 10, wherein the VEGFR-2 antagonist is an antibody, or antigen binding fragment thereof, which comprises: (a) a light chain variable region comprising light chain CDR1, CDR2 and CDR3 of SEQ ID NOs: 17, 18 and 19, respectively, and (b) a heavy chain variable region comprising heavy chain CDR1, CDR2 and CDR3 of SEQ ID NOs: 20, 21 and 22, respectively.

13. The method of any one of claims 1 to 10, wherein the VEGFR-2 antagonist is an anti-VEGFR-2 monoclonal antibody that comprises a heavy chain and a light chain, and wherein the heavy chain comprises a heavy chain variable region comprising SEQ ID NO: 16 and the light chain comprises a light chain variable region comprising SEQ ID NO: 15.

14. The method of any one of claims 1 to 10, wherein the VEGFR-2 antagonist is an anti-VEGFR-2 antibody that comprises a heavy chain and a light chain, and wherein the heavy chain comprises SEQ ID NO: 14 and the light chain comprises SEQ ID NO: 13.

15. The method of any one of claims 1 to 10, wherein the VEGFR-2 antagonist is olinvacimab or an olinvacimab variant.

16. The method of any one of claims 1 to 10, wherein the VEGFR-2 antagonist is ramucirumab.

17. The method of claim 1, wherein the PD-1 antagonist is a humanized anti-PD-1 antibody that comprises a heavy chain and a light chain, and wherein the heavy chain comprises a heavy chain variable region comprising heavy chain CDR1, CDR2 and CDR3 of SEQ ID NOs: 6, 7 and 8, respectively, and the light chain comprises a light chain variable region comprising light chain CDR1, CDR2 and CDR3 of SEQ ID NOs: 1, 2 and 3, respectively; and the VEGFR-2 antagonist is a humanized anti-VEGFR-2 antibody that comprises a heavy chain and a light chain, and wherein the heavy chain comprises a heavy chain variable region comprising heavy chain CDR1, CDR2 and CDR3 of SEQ ID NOs: 20, 21 and 22, respectively, and the light chain comprises a light chain variable region comprising light chain CDR1, CDR2 and CDR3 of SEQ ID NOs: 17, 18 and 19, respectively.

18. The method of claim 1, wherein the PD-1 antagonist is an anti-PD-1 antibody that comprises a heavy chain and a light chain, and wherein the heavy chain comprises a heavy chain variable region comprising SEQ ID NO: 9 and the light chain comprises a light chain variable region comprising SEQ ID NO: 4; and the VEGFR-2 antagonist is an anti-VEGFR-2 antibody that comprises a heavy chain and a light chain, and wherein the heavy chain comprises a heavy chain variable region comprising SEQ ID NO: 16 and the light chain comprises a light chain variable region comprising SEQ ID NO: 15.

19. The method of claim 1, wherein the PD-1 antagonist is an anti-PD-1 antibody that comprises a heavy chain and a light chain, and wherein the heavy chain comprises SEQ ID NO: 10 and the light chain comprises SEQ ID NO: 5; and the VEGFR-2 antagonist is an anti-VEGFR-2 antibody that comprises a heavy chain and a light chain, and wherein the heavy chain comprises SEQ ID NO: 14 and the light chain comprises SEQ ID NO: 13.

20. The method of any one of claims 1 to 19, wherein the PD-1 antagonist and VEGFR-2 antagonist are co-formulated.

21. The method of any one of claims 1 to 19, wherein the PD-1 antagonist and VEGFR-2 antagonist are co-administered.

22. The method of any one of claims 1 to 21, wherein the individual has not been previously treated with anti-PD-1 or anti-PD-L1 therapy or is confirmed progressive while receiving prior anti-PD-1 therapy.

23. The method of claim 1, wherein 200 mg pembrolizumab or a pembrolizumab variant is administered by IV infusion on Day 1 every three weeks, and 16 mg/kg olinvacimab or an olinvacimab variant is administered by IV infusion on Day 1 every week.

24. The method of claim 1, wherein 400 mg pembrolizumab or a pembrolizumab variant is administered on Day 1 every six weeks, and 16 mg/kg olinvacimab or an olinvacimab variant is administered by IV infusion on Day 1 every week for intravenous infusion.

25. The method of any one of claims 1-24, wherein the cancer is triple-negative breast cancer.

26. The method of any one of claims 1-24, wherein the cancer is metastatic triple-negative breast cancer.


SEQUENCE LISTING (23 sequences)

SEQ ID NO: 1 - protein, 15 aa, Artificial Sequence
  CDR1 LC pembrolizumab
RASKGVSTSGYSYLH

SEQ ID NO: 2 - protein, 7 aa, Artificial Sequence
  CDR2 LC pembrolizumab
LASYLES

SEQ ID NO: 3 - protein, 9 aa, Artificial Sequence
  CDR3 LC pembrolizumab
QHSRDLPLT

SEQ ID NO: 4 - protein, 111 aa, Artificial Sequence [sequence omitted]

SEQ ID NO: 5 - protein, 218 aa, Artificial Sequence
  Light Chain pembrolizumab
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, Artificial Sequence
  CDR1 HC pembrolizumab
NYYMY

SEQ ID NO: 7 - protein, 17 aa, Artificial Sequence
  CDR2 HC pembrolizumab
GINPSNGGTNFNEKFKN

SEQ ID NO: 8 - protein, 11 aa, Artificial Sequence
  CDR3 HC pembrolizumab
RDYRFDMGFDY

SEQ ID NO: 9 - protein, 120 aa, Artificial Sequence [sequence omitted]

SEQ ID NO: 10 - protein, 447 aa, Artificial Sequence
  Heavy Chain pembrolizumab
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 213 aa, Artificial Sequence
  Light chain nivolumab
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 12 - protein, 440 aa, Artificial Sequence
  Heavy chain nivolumab
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 227 aa, Artificial Sequence
  Light Chain olinvacimab
SGVGSNFMLTQPPSVSVSPGKTARITCRGDNLGDVNVHWYQQRPGQAPVLVMYYDADRPSGIPERFSGSNSGNTATLTISGVEAGDEADYYCQVWDRTSEYVFGTGTKVTVLGGGASLVERSVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 462 aa, Artificial Sequence
  Heavy Chain olinvacimab
AQPAMAQMQLVQSGAEVKKPGASVKLSCKASGYTFSSYWMHWVRQAPGQRLEWMGEINPGNGHTNYNEKFKSRVTITVDKSASTAYMELSSLRSEDTAVYYCAKIWGPSLTSPFDYWGQGTLVTVSSGLGGLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 108 aa, Artificial Sequence
  LC Variable Region olinvacimab
NFMLTQPPSVSVSPGKTARITCRGDNLGDVNVHWYQQRPGQAPVLVMYYDADRPSGIPERFSGSNSGNTATLTISGVEAGDEADYYCQVWDRTSEYVFGTGTKVTVLG

SEQ ID NO: 16 - protein, 121 aa, Artificial Sequence
  HC Variable Region olinvacimab
QMQLVQSGAEVKKPGASVKLSCKASGYTFSSYWMHWVRQAPGQRLEWMGEINPGNGHTNYNEKFKSRVTITVDKSASTAYMELSSLRSEDTAVYYCAKIWGPSLTSPFDYWGQGTLVTVSS

SEQ ID NO: 17 - protein, 11 aa, Artificial Sequence
  CDR-L1 olinvacimab
RGDNLGDVNVH

SEQ ID NO: 18 - protein, 7 aa, Artificial Sequence
  CDR-L2 olinvacimab
YDADRPS

SEQ ID NO: 19 - protein, 10 aa, Artificial Sequence
  CDR-L3 olinvacimab
QVWDRTSEYV

SEQ ID NO: 20 - protein, 5 aa, Artificial Sequence
  CDR-H1 olinvacimab
SYWMH

SEQ ID NO: 21 - protein, 17 aa, Artificial Sequence
  CDR-H2 olinvacimab
EINPGNGHTNYNEKFKS

SEQ ID NO: 22 - protein, 12 aa, Artificial Sequence
  CDR-H3 olinvacimab
IWGPSLTSPFDY

SEQ ID NO: 23 - protein, 327 aa, Homo sapiens
  MISC_FEATURE (1)..(327): amino acids 1-327 of Uniprot accession no. P35968
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK